(19) United States
(12) Patent Application Publication
(10) Pub. No.: US 2008/0063650 A1
(43) Pub. Date: Mar. 13, 2008

(54) mCRP antagonists and their uses

(76) Inventor: Jun Yan, Louisville, KY (US)

Correspondence Address: Biopolymer Engineering, Inc. dba Biothera, Inc.;c/o Intellevate, P.O. Box 52050, Minneapolis, MN, 55402, US

(21) Appl. No.: 11/899,022

(22) Filed: Sep. 4, 2007

Related U.S. Application Data
(60) Provisional application No. 60/841,849, filed on Sep. 1, 2006.

(51) Int. Cl. A61K 39/395; A61K 31/716; A61P 43/00

(52) U.S. Cl. 424/172.1; 424/130.1; 514/54

(57) ABSTRACT

A therapeutic composition for treating a proliferative disorder includes a membrane-bound complement regulatory protein (mCRP) antagonist, a therapeutic ligand that is capable of activating complement and β-glucan. mCRPs are overexpressed in some tumor types and are shown to inhibit complement activation. mCRP antagonists inhibit or block mCRP activity such that the therapeutic ligand and β-glucan effectively treat the proliferative disorder.

This application claims the benefit of U.S. Ser. No. 60/841,849 entitled mCRP ANTAGONISTS AND THEIR USES, filed on Sep. 1, 2006.

### BACKGROUND OF THE INVENTION

The present invention relates to agents that block membrane-bound complement regulatory protein (mCRP) function and their methods of use. More particularly, the present invention relates to protein antagonists of mCRPs and their use in enhancing the activity of anti-proliferative/β-glucan immunotherapy.

β-glucan is a complex carbohydrate derived from sources including yeast and other fungi, bacteria and cereal grains. The potential antitumor activity of β-glucans has been under extensive investigation. The effectiveness of various glucan preparations has differed in their ability to elicit various cellular responses, particularly cytokine expression and production, and in their activity against specific tumors.

The immune system comprises two overall systems, the adaptive immune system and the innate immune system. β-glucans are generally thought to operate through the innate immune system, which includes complement proteins, macrophages, neutrophils and natural killer (NK) cells. It serves as a rapid means of dealing with infection before the adaptive immune system takes effect.

In many cases, subtle changes associated with cancer or other proliferative disorders can lead to altered expression of surface proteins. These changes stimulate a weak response of the adaptive immune system and may provide a target for treatment using selective mAbs or antitumor vaccines. Suppression and eradication of cancer cells opsonized with antibody may occur via antibody-dependent cellular cytotoxicity (ADCC) and/or complement-dependent cytotoxicity (CDC). In addition, iC3b deposited on the surface of tumor cells during complement activation binds complement receptor 3 (CR3, CD11b/CD18, Mac1), which may enhance ADCC or induce effector cells to carry out cellular degranulation (CR3-dependent cellular cytotoxicity).

Soluble β-glucan that binds to the lectin site of CR3 primes circulating phagocytes and natural killer (NK) cells, permitting cytotoxic degranulation in response to iC3b-opsonized tumor cells that otherwise escape from this mechanism of cell-mediated cytotoxicity. CR3 binds soluble β-glucan with high affinity (5×10⁻⁸ M). The tumoricidal response promoted by soluble β-glucan in mice was shown to be absent in mice deficient in either serum C3 (complement 3) or leukocyte CR3, highlighting the requirement for iC3b on tumors and CR3 on leukocytes in the tumoricidal function of β-glucans. Vetvicka, V., et al., J. Clin. Invest. 98:50-61 (1996) and Yan, J., V. et al., J. Immunol. 163:3045-3052 (1999)).

Complement plays a very important role in the antitumor activity of β-glucan. When complement C3b has attached itself to a surface, it may be clipped by a serum protein to produce a smaller fragment, iC3b. While iC3b has been “inactivated” and cannot function to form a membrane attack complex, it remains attached to the surface where it serves to attract neutrophils and macrophages which can phagocytize or otherwise destroy the marked (“opsonized”) cell. On the surface of neutrophils and macrophages are complement receptors (CR3) that bind to iC3b.

### SUMMARY OF THE INVENTION

The present invention is a therapeutic composition to a proliferative disorder. The therapeutic composition includes a mCRP antagonist to maintain at least some complement activity, a therapeutic ligand that is specific to markers or antigens related to the proliferative disorder and β-glucan. The present invention also encompasses treatments and kits utilizing the therapeutic compositions.

### BRIEF DESCRIPTION OF THE DRAWINGS

FIG. 1 shows graphical representations of flow cytometry results indicating the presence of CD46, CD55 and CD59 on NCI-H23 and SKOV-3 cells.

FIG. 2 shows microscopic images of CD55 expression on NCI-H23 and SKOV-3 tumor cells.

FIG. 3A shows microscopic images of neutrophil infiltration in NCI-H23 and SKOV-3 tumors.

FIG. 3B graphically shows the average number of neutrophils present per high power microscope field in NCI-H23 and SKOV-3 tumors.

FIG. 4A shows microscopic images of macrophage infiltration in NCI-H23 and SKOV-3 tumors.

FIG. 4B graphically shows the average number of macrophages present per high power microscope field in NCI-H23 and SKOV-3 tumors.

FIG. 5 shows microscopic images of C5a deposition in NCI-H23 and SKOV-3 cells.

FIG. 6 graphically shows flow cytometry results indicating that blocking CD46 or CD55 on Herceptin opsinized SKOV-3 increases human or mouse iC3b deposition.

### DETAILED DESCRIPTION OF THE INVENTION

Tumor immunotherapy with humanized mAbs is now accepted clinical practice. Examples of such mAbs include Herceptin™ (trastuzumab) and Rituxan™ (rituximab) for patients with Her-2/neu⁺ metastatic breast mammary carcinoma and B cell lymphoma, respectively, and Erbitux™ (cetuximab) for patients with over-expressed EGFR colon or rectal cancers. Unfortunately, antibody immunotherapy is not uniformly effective.

Several factors have been considered as causes for the non-uniform results. In vitro studies have shown that complement-dependent cytotoxicity (CDC) is limited by membrane-bound complement regulatory proteins (mCRPs) that are overexpressed on some tumors. Some of these factors include CD46, CD55 and CD59.

CD46 (membrane cofactor protein, MCP) inhibits C3b activity. It serves as a receptor for seven human pathogens but was initially discovered as a widely expressed C3b- and C4b-binding protein. It was later shown to be a cofactor for the serine protease factor I to inactivate by limited proteolysis C3b- and C4b-binding proteins and components of the convertases. CD46 is additionally a protector of placental tissue and is expressed on the inner acrosomal membrane of spermatozoa. Cross-linking CD46 with antibodies or natural or pathogenic ligands induces rapid turnover and signaling events. It has an important role in protecting cells from excessive complement activation and even a heterozygous deficiency of CD46 predisposes individuals to hemolytic uremic syndrome.

CD55 (decay accelerating factor, DAF) inhibits C3 convertases. It was first recognized as a species restricting factor operating at the level of C3 activation. It binds C3b and C4b to inhibit formation and half-life of the C3 convertases. It is also a receptor for echovirus and Coxsackie B virus. Like CD46, evidence indicates CD55 is a ligand or protective molecule in fertilization. CD55 is broadly distributed among cells in contact with serum, including both hematopoietic and non-hematopoietic cells. Expression on NK cells or their targets has been associated with reduced efficiency of cell lysis. Although CD55 does not have an essential role in controlling hemolysis of erythrocytes, it has an important role in regulation of the deposition of C3 on nucleated cells.

CD59 (IF-5Ag, H19, HRF20, MACIF, MIRL, P-18, Protectin) inhibits membrane attack complex (MAC) formation. CD59 blocks the formation of MAC by preventing development of a hydrophobic region on the C9 molecule, important for insertion into the target cell membrane. A single C9 may bind to the C5b-8 complex but it cannot be unfolded to allow binding of multiple subsequent C9 molecules. Therefore, MAC formation is terminated and lysis of the target cell does not occur.

In addition, CD59 prevents ion channel formation by the C5b-8 complex and the small-size MAC C5b-C9 complex formed by attachment of a single C9 molecule, which in turn prevents leaky pore formation. CD59 is critically important in reducing the lytic effect of C5b-8 or C5b-9 during the time interval before the target cell is able to remove the MAC complex by shedding, vesiculation or endocytosis.

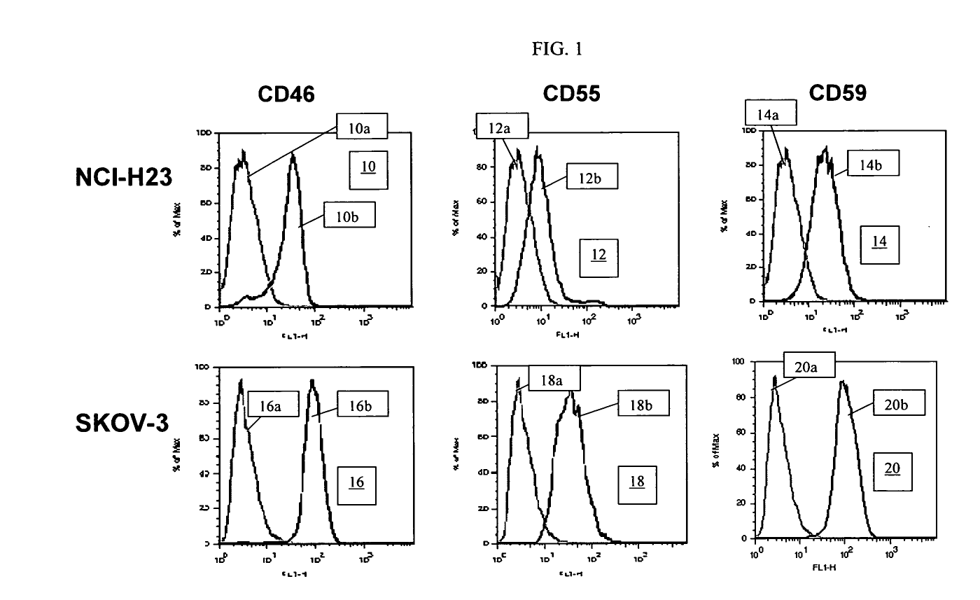

The correlation between increased mCRP expression and decreased immunotherapy effectiveness is illustrated in FIG. 1. Cells' from two tumor cell lines, NCI-H23 and SKOV-3, were stained with either anti-CD46, anti-CD55 or anti-CD59 fluorescently tagged antibodies and subjected to flow cytometry. NCI-H23 is a human lung carcinoma cell line and SKOV-3 is a human ovarian carcinoma cell line. In general, NCI-H23 tumors respond more favorably to immunotherapy than SKOV-3 tumors.

Results are shown in graphs 10-20 of FIG. 1. Graphs 10, 12 and 14 illustrate increased expression of CD46, CD55 and CD59, respectively, on NCI-H23 cells relative to isotype antibody control staining. Histograms 10a, 12a and 14a represent isotype control antibody staining, while histograms 10b, 12b and 14b represent mCRP expression on lung tumor cells. As is evident from the graphs, there is an increase in mCRP expression on the tumor cells.

Graphs 16, 18 and 20 illustrate increased expression of CD46, CD55 and CD59, respectively, on SKOV-3 cells relative to isotype antibody control staining. Histograms 16a, 18a and 20a represent isotype control antibody staining, while histograms 16b, 18b and 20b represent mCRP expression on ovarian tumor cells. There is a significant increase in mCRP expression on the SKOV-3 cells, even over that expressed on the NCI-H23 cells.

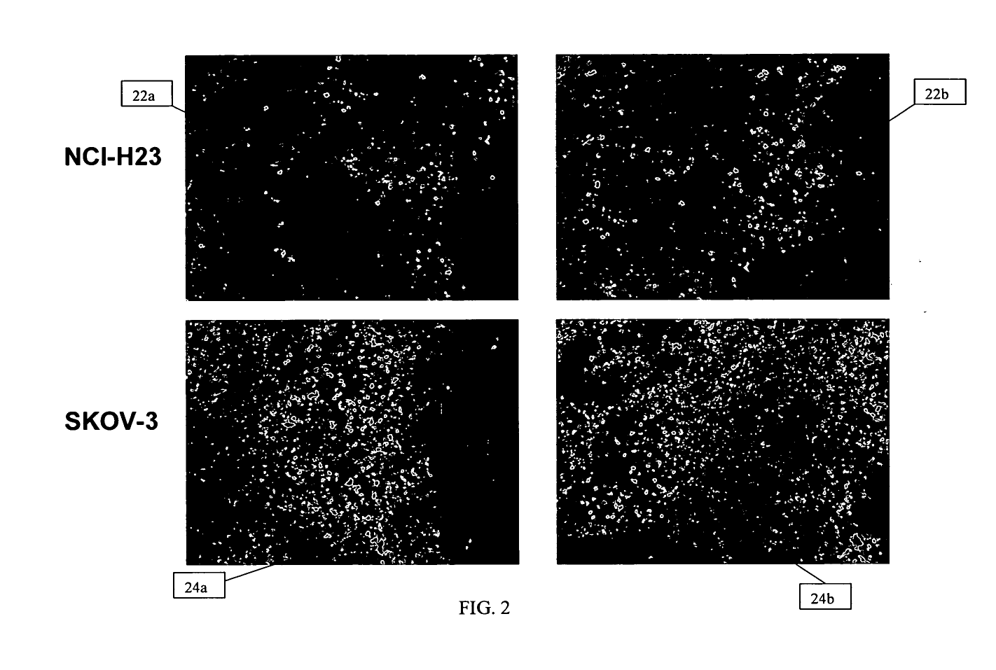

Results of the above flow cytometry experiments were confirmed microscopically. An example is shown in FIG. 2. Here, NCI-H23 and SKOV-3 tumor specimens were stained with fluorescently tagged anti-CD55 Abs and examined microscopically. Slides 22a and 22b are representative fields showing CD55 expression on NCI-H23 cells. Slides 24a and 24b are representative fields showing CD55 expression on SKOV-3 cells. Here again, SKOV-3 cells show a significant increase in CD55 expression relative to NCI-H23 cells.

As previously discussed, mCRPs such as CD46 and CD55 effect early steps in the complement cascade, which then ultimately also effect C5a formation. C5a is an anaphylatoxin and has a chemotactic effect on granulocytes, monocytes, and macrophages, all of which have receptors for C5a (CD88).

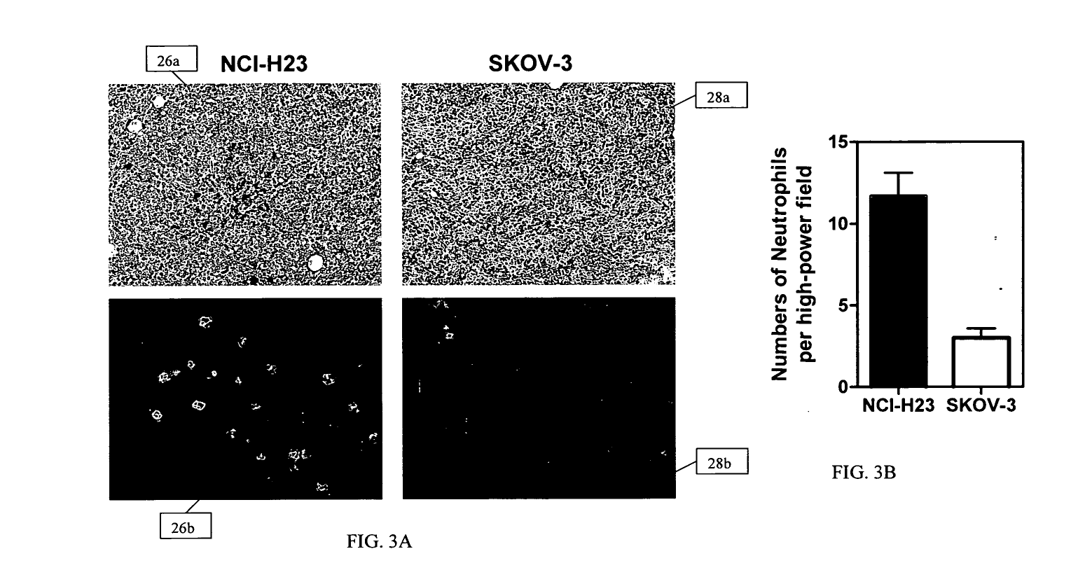

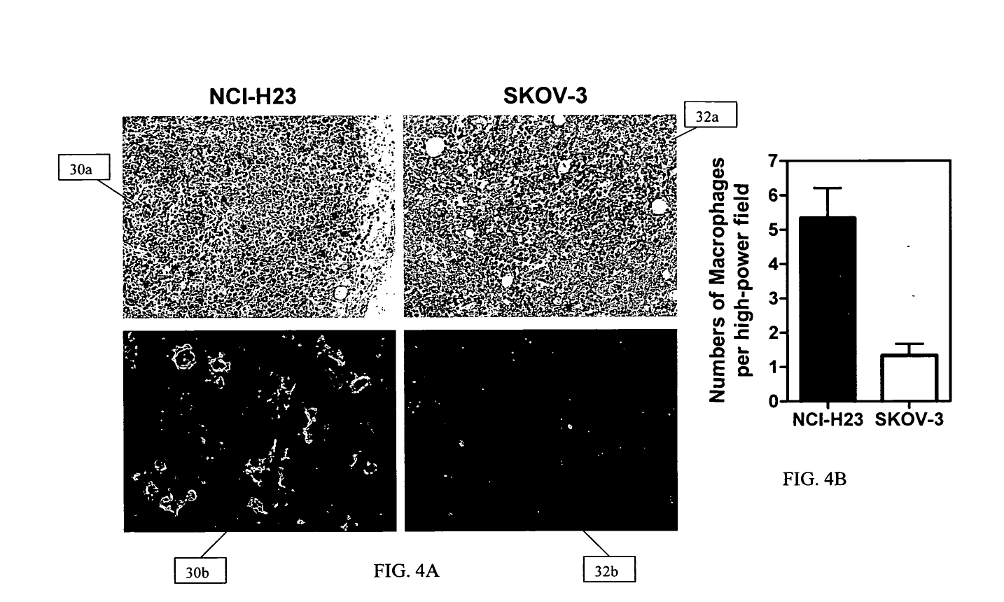

The relative amounts of neutrophil and macrophage infiltration in lung and ovarian tumors are shown in FIGS. 3A and 3B and FIGS. 4A and 4B, respectively. Slides 26a and 26b of FIG. 3A are representative fields showing non-fluorescent and fluorescent staining of neutrophils, respectively, in NCI-H23 tumors. Slides 28a and 28b are similarly stained fields in SKOV-3 tumors. The difference in neutrophil infiltration between the two tumor types is significant with the SKOV-3 tumors having a marked decrease in detectable neutrophils.

The average number of neutrophils per field was calculated, and the results are given in the bar graph of FIG. 3B. NCI-H23 tumors are populated with about four times more neutrophils than SKOV-3 tumors.

Slides 30a and 30b of FIG. 4A are representative fields showing non-fluorescent and fluorescent staining of macrophages, respectively, in NCI-H23 tumors. Slides 32a and 32b are similarly stained fields in SKOV-3 tumors. As with neutrophil infiltration, macrophage infiltration is significantly reduced in the SKOV-3 tumors.

The average number of macrophages per field was calculated for the two tumor types, and the results are shown in the bar graph of FIG. 4B. NCI-H23 tumors are populated with about 3.5 times more macrophages than SKOV-3 tumors.

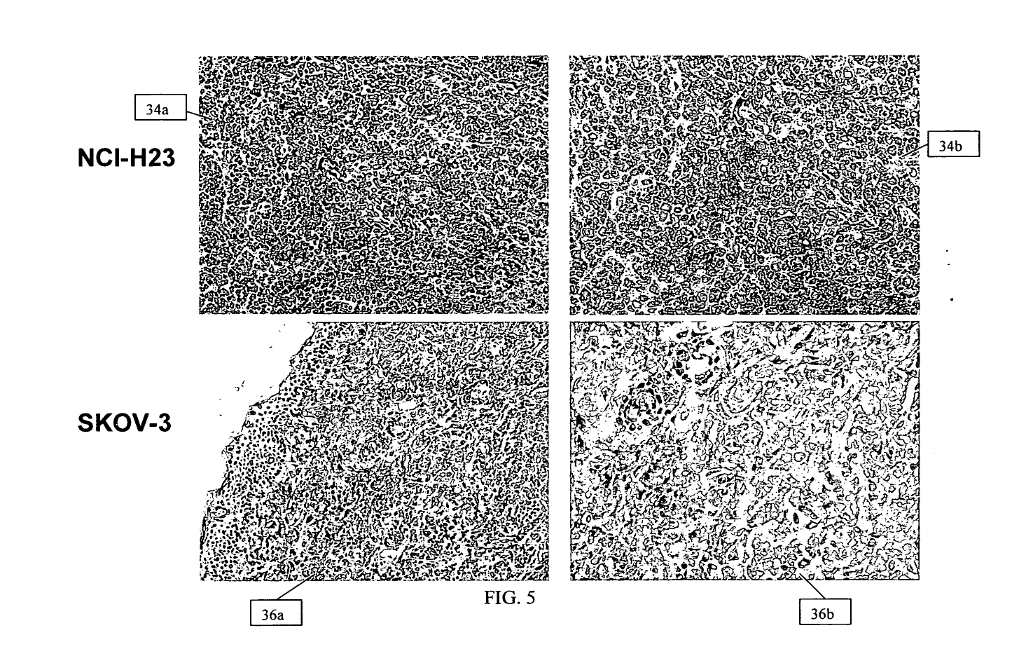

As suggested above, decreased neutrophil and macrophage infiltration is likely linked to decreased C5a deposition. Reduced C5a deposition within the tumor types is shown in FIG. 5. Slides 34a and 34b show C5a deposition in NCI-H23 cells, and slides 36a and 36b show C5a deposition in SKOV-3 cells. As is evident, production and binding of C5a is markedly reduced in SKOV-3 tumors relative to the NCI-H23 tumors.

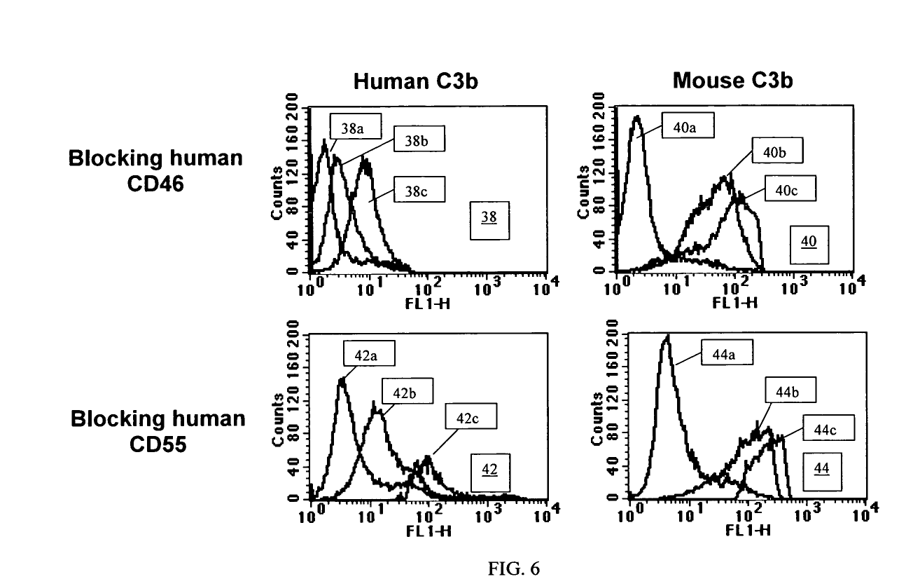

The above results suggest that the increased expression of mCRPs in certain tumor cells inhibits complement activation. This was confirmed by blocking CD46 and CD55 in SKOV-3 cells opsonized with Herceptin. Specifically, SKOV-3 cells were incubated with Herceptin and anti-human CD46 or anti-human CD55 Abs. The cells were subsequently exposed to complement, which, if activated, results in iC3b deposition on the cell surface. The amount of bound iC3b was measured via labeled anti-iC3b Ab. The results are shown in FIG. 6.

Graph 38 includes histograms 38a, 38b and 38c showing the effect of CD46 blockage on human complement activation. Histogram 38a represents control cells, which were not exposed to Ab but were exposed to human complement. Histogram 38b represents a second control where cells were exposed to Herceptin and human complement but were not exposed to anti-human CD46 Ab. Histogram 38c represents cells exposed to Herceptin, anti-human CD46 Ab and human complement. The shift of histogram 38c indicates that CD46 blocking resulted in a marked increase in complement activation.

Graph 40 shows the results of a similar experiment except carried out with mouse complement instead of human complement. Histograms 40a, 40b and 40c represent similarly treated cells as those described for histograms 38a, 38b and 38c, respectively. Again, CD46 blocking increased complement activation.

Graph 42 includes histograms 42a, 42b and 42c showing the effect of CD55 blockage on human complement activation. Control and experimental cells represented by histograms 42a, 42b and 42c were treated in a similar manner as those described for histograms 38a, 38b and 38c, respectively, except that anti-human CD55 Ab was included instead of anti-human CD46 Ab. Graph 42 shows that CD55 blocking significantly increases human complement activation.

Similarly, graph 44 shows increased activation of mouse complement with CD55 blocking. Histograms 44a, 44b and 44c are representative of cells treated similarly to those described for histograms 42a, 42b and 42c, respectively.

As previously discussed, β-glucan can increase the effectiveness of anti-cancer, or anti-proliferative, immunotherapy as long as complement activation is not inhibited leading to iC3b deposition on tumor cells. Conversely, the decreased effectiveness of an anti-cancer immunotherapy cannot be fully compensated for with therapeutic administration of β-glucan if complement activation is essentially blocked. Therefore, the activity of overexpressed mCRPs should be decreased or inhibited in order to restore at least some complement functionality and maintain the effectiveness of β-glucan/anti-cancer Ab immunotherapy.

Decreasing or inhibiting mCRP activity may be accomplished with any type of mCRP antagonist. The experiments described above show that anti-mCRP Abs provide one means of inhibiting mCRP activity. mCRP antagonists used in the composition of the present invention include, for example, polyclonal and monoclonal antibodies, recombinant chimeric monoclonal antibodies, antibody fragments, other proteins and small molecules that bind specifically to mCRP domains.

The use of mAbs as mCRP antagonists has some limitations. mAbs are large molecules that cannot penetrate into target tumor tissues. In addition, they cannot be administered orally and must be given in concentrations in the order of 5,000-10,000 times the concentration of their targets to be effective. Furthermore, mAbs must be produced in cell culture, making them relatively expensive to manufacture. Nevertheless, certain characteristics of mAbs, such as high specificity accompanied by low toxicity, make them desirable mCRP antagonists and should be conserved.

Small protein/peptide molecules that act as mCRP antagonists are a suitable alternative and may be generated by computer-modeling software that designs these molecules to mimic anti-mCRP Abs such as anti-CD46, anti-CD55 and anti-CD59. Computer modeling is used to design protein/peptide mCRP antagonists. A suitable small peptide mCRP antagonist could be as small as about 15 to about 20 amino acids in length but may be larger. Their relative small size allows for better penetration of tumor tissues and makes them more stable and easier to manufacture. In addition, small peptide mCRP antagonists or suppressive oligodeoxynucleotide (ODNS) or siRNA (RNA interference) can be engineered to a desired specificity and affinity for a chosen mCRP. Any of the above-mentioned mCRP antagonists can be administered separately or in various combinations.

Complement activating ligands such as antibodies (both naturally found or produced by methods known in the art) are directed to cellular markers or antigens and are able to activate one or more members of the complement cascade. In other words, a ligand that activates complement sufficiently to deposit iC3b on the tumor cells is needed. In certain embodiments, antagonists include IgG subclass 1 or IgG subclass 2 Abs.

The β-glucans used in the invention include PGG (poly-(1-6)-β-D-glucopyranosyl-(1-3)-β-D-glucopyranose), neutral soluble β-glucan, triple helical β-glucan (BETAFECTIN™), β-glucans of various aggregate numbers and an active 25 kDa β-glucan. The above-mentioned species of β-glucans are administered separately or in various combinations. The β-glucan preparations of this invention are prepared from insoluble glucan particles. The glucan described herein can be made by various methods known to one skilled in the art. For example, the preparation of neutral soluble glucan (NSG) is described in U.S. Pat. No. 5,322,841, the disclosure of which is incorporated herein by reference. Briefly, this method involves treating whole glucan particles with a series of acid and alkaline treatments to produce soluble glucan that forms a clear solution at a neutral pH. The whole glucan particles utilized in this present invention can be in the form of a dried powder, prepared by the process described above.

Whole glucan particles are suspended in an acid solution under conditions sufficient to dissolve the acid-soluble glucan portion. For most glucans, an acid solution having a pH of from about 1 to about 5 and a temperature of from about 20° to about 100° C. is sufficient. Typically, the acid used is an organic acid capable of dissolving the acid-soluble glucan portion. Acetic acid, at concentrations of from about 0.1 to about 5M or formic acid at concentrations of from about 50% to 98% (w/v) are useful for this purpose. Additionally, sulphuric acid can be utilized. The treatment is usually carried out at about 90° C. The treatment time may vary from about 1 hour to about 20 hours depending on the acid concentration, temperature and source of whole glucan particles. For example, modified glucans having more β(1-6) branching than naturally-occurring, or wild-type glucans, require more stringent conditions, i.e., longer exposure times and higher temperatures. This acid-treatment step can be repeated under similar or variable conditions. In one embodiment of the present method, modified whole glucan particles from the strain, S. cerevisiae R4, which have a higher level of β(1-6) branching than naturally-occurring glucans, are used, and treatment is carried out twice: first with 0.5M acetic acid at 90° C. for 3 hours and second with 0.5M acetic acid at 90° C. for 20 hours.

The acid-insoluble glucan particles are then separated from the solution by an appropriate separation technique, for example, by centrifugation or filtration. The pH of the resulting slurry is adjusted with an alkaline compound such as sodium hydroxide, to a pH of about 7 to about 14. The slurry is then resuspended in hot alkali having a concentration and temperature sufficient to solubilize the glucan polymers. Alkaline compounds which can be used in this step include alkali-metal or alkali-earth metal hydroxides, such as sodium hydroxide or potassium hydroxide, having a concentration of from about 0.1 to about 10N. This step can be conducted at a temperature of from about 4° C. to about 121° C., typically from about 20° C. to about 100° C. In one embodiment of the process, the conditions utilized are a 1 N solution of sodium hydroxide at a temperature of about 80°-100° C. and a contact time of approximately 1-2 hours. The resulting mixture contains solubilized glucan molecules and particulate glucan residue and generally has a dark brown color due to oxidation of contaminating proteins and sugars. The particulate residue is removed from the mixture by an appropriate separation technique, e.g., centrifugation and/or filtration.

The resulting solution contains soluble glucan molecules. This solution can, optionally, be concentrated to effect a 5 to 10 fold concentration of the retentate soluble glucan fraction to obtain a soluble glucan concentration in the range of about 1 to 5 mg/ml. This step can be carried out by an appropriate concentration technique, for example, by ultrafiltration, utilizing membranes with nominal molecular weight levels.

The concentrated fraction obtained after this step is enriched in the soluble, biologically active glucan, also referred to as β-glucan PGG. To obtain a pharmacologically acceptable solution, the glucan concentrate is further purified, for example, by diafiltration. In one embodiment, diafiltration is carried out using approximately 10 volumes of alkali in the range of about 0.2 to 0.4N. A suitable concentration of the soluble glucan after this step is from about 2 to about 5 mg/ml. The pH of the solution is adjusted in the range of about 7-9 with an acid, such as hydrochloric acid. Traces of proteinaceous material, which may be present can be removed by contacting the resulting solution with a positively charged medium such as DEAE-cellulose, QAE-cellulose or Q-Sepharose.

The highly purified, glucan solution can be further purified, for example, by diafiltration, using a pharmaceutically acceptable medium (e.g., sterile water for injection, phosphate-buffered saline (PBS), isotonic saline, dextrose) suitable for parenteral administration. The final concentration of the glucan solution is adjusted in the range of about 0.5 to 5 mg/ml. In accordance with pharmaceutical manufacturing standards for parenteral products, the solution can be terminally sterilized by filtration through a 0.22 μm filter. The soluble glucan preparation obtained by this process is sterile, non-antigenic, and essentially pyrogen-free, and can be stored at room temperature for extended periods of time without degradation.

Alternative methods for preparing particulate and soluble β-glucan are described in U.S. Appln. No. 60/813,971, which is herein incorporated by reference and briefly described. A yeast culture is grown, typically, in a shake flask or fermenter. In one embodiment of bulk production, a culture of yeast is started and expanded stepwise through a shake flask culture into a 250-L scale production fermenter. The yeast are grown in a glucose-ammonium sulfate medium enriched with vitamins, such as folic acid, inositol, nicotinic acid, pantothenic acid (calcium and sodium salt), pyridoxine HCl and thymine HCl and trace metals from compounds such as ferric chloride, hexahydrate; zinc chloride; calcium chloride, dihydrate; molybdic acid; cupric sulfate, pentahydrate and boric acid. An antifoaming agent such as Antifoam 204 may also be added at a concentration of about 0.02%.

The production culture is maintained under glucose limitation in a fed batch mode. During seed fermentation, samples are taken periodically to measure the optical density of the culture before inoculating the production fermenter. During production fermentation, samples are also taken periodically to measure the optical density of the culture. At the end of fermentation, samples are taken to measure the optical density, the dry weight, and the microbial purity.

If desired, fermentation may be terminated by raising the pH of the culture to at least 11.5 or by centrifuging the culture to separate the cells from the growth medium. In addition, depending on the size and form of purified β-glucan that is desired, steps to disrupt or fragment the yeast cells may be carried out. Any known chemical, enzymatic or mechanical methods, or any combination thereof may be used to carry out disruption or fragmentation of the yeast cells.

The yeast cells containing the β-glucan are harvested. When producing bulk β-glucan, yeast cells are typically harvested using continuous-flow centrifugation.

Yeast cells are extracted utilizing one or more of an alkaline solution, a surfactant, or a combination thereof. A suitable alkaline solution is, for example, 0.1 M-5 M NaOH. Suitable surfactants include, for example, octylthioglucoside, Lubrol PX, Triton X-100, sodium lauryl sulfate (SDS), Nonidet P-40, Tween 20 and the like. Ionic (anionic, cationic, amphoteric) surfactants (e.g., alkyl sulfonates, benzalkonium chlorides, and the like) and nonionic surfactants (e.g., polyoxyethylene hydrogenated castor oils, polyoxyethylene sorbitol fatty acid esters, polyoxyethylene sorbitan fatty acid esters, polyoxyethylene glycerol fatty acid esters, polyethylene glycol fatty acid esters, polyoxyethylene alkyl phenyl ethers, and the like) may also be used. The concentration of surfactant will vary and depend, in part, on which surfactant is used. Yeast cell material may be extracted one or more times.

Extractions are usually carried out at temperatures between about 70° C. and about 90° C. Depending on the temperature, the reagents used and their concentrations, the duration of each extraction is between about 30 minutes and about 3 hours.

After each extraction, the solid phase containing the β-glucan is collected using centrifugation or continuous-flow centrifugation and resuspended for the subsequent step. The solubilized contaminants are removed in the liquid phase during the centrifugations, while the β-glucan remains in the insoluble cell wall material.

In one embodiment, four extractions are carried out. In the first extraction, harvested yeast cells are mixed with 1.0 M NaOH and heated to 90° C. for approximately 60 minutes. The second extraction is an alkaline/surfactant extraction whereby the insoluble material is resuspended in 0.1 M NaOH and about 0.5% to 0.6% Triton X-100 and heated to 90° C. for approximately 120 minutes. The third extraction is similar to the second extraction except that the concentration of Triton X-100 is about 0.05%, and the duration is shortened to about 60 minutes. In the fourth extraction, the insoluble material is resuspended in about 0.05% Triton-X 100 and heated to 75° C. for approximately 60 minutes.

The alkaline and/or surfactant extractions solubilize and remove some of the extraneous yeast cell materials. The alkaline solution hydrolyzes proteins, nucleic acids, mannans, and lipids. Surfactant enhances the removal of lipids, which provides an additional advantage yielding an improved β-glucan product.

The next step in the purification process is an acidic extraction to remove glycogen. One or more acidic extractions are accomplished by adjusting the pH of the alkaline/surfactant extracted material to between about 5 and 9 and mixing the material in about 0.05 M to about 1.0 M acetic acid at a temperature between about 70° C. and 100° C. for approximately 30 minutes to about 12 hours.

In one embodiment, the insoluble material remaining after centrifugation of the alkaline/surfactant extraction is resuspended in water, and the pH of the solution is adjusted to about 7 with concentrated HCl. The material is mixed with enough glacial acetic acid to make a 0.1 M acetic acid solution, which is heated to 90° C. for approximately 5 hours.

Next, the insoluble material is washed. In a typical wash step, the material is mixed in purified water at about room temperature for a minimum of about 20 minutes. The water wash is carried out two times. The purified β-glucan product is then collected. Again, collection is typically carried out by centrifugation or continuous-flow centrifugation.

At this point, a purified, particulate β-glucan product is formed. The product may be in the form of whole glucan particles or any portion thereof, depending on the starting material. In addition, larger sized particles may be broken down into smaller particles by any chemical, enzymatic or mechanical means. The range of product sizes includes β-glucan particles that have substantially retained in vivo morphology (whole glucan particles) down to submicron-size particles.

As is well known in the art, particulate β-glucan is useful in many food, supplement and pharmaceutical applications. Alternatively, particulate β-glucan can be processed further to form aqueous, soluble β-glucan.

Particuate β-glucan starting material may range in size from whole glucan particles down to submicron-sized particles. The particulate β-glucan undergoes an acidic treatment under pressure and elevated temperature to produce soluble β-glucan. Pelleted, particulate β-glucan is resuspended and mixed in a sealable reaction vessel in a buffer solution and brought to pH 3.6. Buffer reagents are added such that every liter, total volume, of the final suspension mixture contains about 0.61 g sodium acetate, 5.24 ml glacial acetic acid and 430 g pelleted, particulate β-glucan. The vessel is purged with nitrogen to remove oxygen and increase the pressure within the reaction vessel.

In a particular embodiment, the pressure inside the vessel is brought to 35 PSI, and the suspension is heated to about 135° C. for between about 4.5 and 5.5 hours. It was found that under these conditions the β-glucan will solubilize. As the temperature decreases from 135° C., the amount of solubilization also decreases.

It should be noted that this temperature and pressure are utilized in a particular embodiment. Optimization of temperatures and pressures may be required depending on reaction conditions and/or reagents.

The exact duration of heat treatment is typically determined experimentally by sampling reactor contents and performing gel permeation chromatography (GPC) analyses. The objective is to maximize the yield of soluble material that meets specifications for high resolution-GPC(HR-GPC) profile and impurity levels, which are discussed below. Once the β-glucan is solubilized, the mixture is cooled to stop the reaction.

The crude, solubilized β-glucan may be washed and utilized in some applications at this point, however, for pharmaceutical applications further purification is performed. Any combination of one or more of the following steps may be used to purify the soluble β-glucan. Other means known in the art may also be used if desired. First, the soluble β-glucan is clarified. Suitable clarification means include, for example, centrifugation or continuous-flow centrifugation.

Next, the soluble β-glucan may be filtered. In one embodiment, the material is filtered, for example, through a depth filter followed by a 0.2 μm filter.

Chromatography may be used for further purification. The soluble β-glucan may be conditioned at some point during previous steps in preparation for chromatography. For example, if a chromatographic step includes hydrophobic interaction chromatography (HIC), the soluble β-glucan can be conditioned to the appropriate conductivity and pH with a solution of ammonium sulphate and sodium acetate. A suitable solution is 3.0 M ammonium sulfate, 0.1 M sodium acetate, which is used to adjust the pH to 5.5.

In one example of HIC, a column is packed with Tosah Toyopearl Butyl 650M resin (or equivalent). The column is packed and qualified according to the manufacturer's recommendations.

Prior to loading, the column equilibration flow-through is sampled for pH, conductivity and endotoxin analyses. The soluble β-glucan, conditioned in the higher concentration ammonium sulphate solution, is loaded and then washed. The nature of the soluble β-glucan is such that a majority of the product will bind to the HIC column. Low molecular weight products as well as some high molecular weight products are washed through. Soluble β-glucan remaining on the column is eluted with a buffer such as 0.2 M ammonium sulfate, 0.1 M sodium acetate, pH 5.5. Multiple cycles may be necessary to ensure that the hexose load does not exceed the capacity of the resin. Fractions are collected and analyzed for the soluble β-glucan product.

Another chromatographic step that may be utilized is gel permeation chromatography (GPC). In one example of GPC, a Tosah Toyopearl HW55F resin, or equivalent, is utilized and packed and qualified as recommended by the manufacturer. The column is equilibrated and eluted using citrate-buffered saline (0.14 M sodium chloride, 0.011 M sodium citrate, pH 6.3). Prior to loading, column wash samples are taken for pH, conductivity and endotoxin analyses. Again, multiple chromatography cycles may be needed to ensure that the load does not exceed the capacity of the column.

The eluate is collected in fractions, and various combinations of samples from the fractions are analyzed to determine the combination with the optimum profile. For example, sample combinations may be analyzed by HR-GPC to yield the combination having an optimized HR-GPC profile. In one optimized profile, the amount of high molecular weight (HMW) impurity, that is soluble β-glucans over 380,000 Da, is less than or equal to 10%. The amount of low molecular weight (LMW) impurity, under 25,000 Da, is less than or equal to 17%. The selected combination of fractions is subsequently pooled.

At this point, the soluble β-glucan is purified and ready for use. Further filtration may be performed in order to sterilize the product. If desired, the hexose concentration of the product can be adjusted to about 1.0±0.15 mg/ml with sterile citrate-buffered saline.

An active 25 kD β-glucan may also be utilized with the present invention. It is described in U.S. Appln. No. 60/814,148, which is herein incorporated by reference. Briefly, the 25 kD β-glucan is the product of macrophage processing of a parent β-glucan. To prepare the 25 kD β-glucan, macrophages are maintained in a bioreactor flask in macrophage growth serum-free medium, such as SFM medium, Invitrogen, Grand Island, N.Y. Labeled soluble β-glucan is added to the culture. After about three weeks of cell culture, the cell-free fluid from the lower chamber of the bioreactor flask containing soluble fragments of β-glucan is collected. The β-glucan fragments are separated by, for example, high-performance liquid chromatography (HPLC) (Waters 1525, Waters Corp., Milford, Mass.) utilizing a monophasic gradient and separated on a Sephacryl S-200 (GE Healthcare, formerly Amersham Biosciences, Piscataway, N.J.) column. Dextran standards of known molecular weights establish a molecular weight elution profile. Fractions containing labeled material corresponding to a molecular weight of 25 kD, as detected by an appropriate detection method, are collected. The 25 kD β-glucan may be further purified and concentrated by ultracentrifugation with, for example, a Centriprep (Millipore Corp., Bedford, Mass.) with a 50 kD cutoff membrane.

The invention also encompasses a method of treating a proliferative disorder, such as cancer or microbial infections including intracellular infections, in a mammal by administering to the mammal a composition including a β-glucan, a therapeutic ligand and a mCRP antagonist. The method may further comprise the administration of other anti-cancer or anti-microbial drugs with the β-glucan, therapeutic ligand and an mCRP antagonist. The invention also encompasses a method of treating an immune dysfunction in a mammal by administering to the mammal a composition including a O-glucan, a therapeutic ligand and a mCRP antagonist.

The compositions may, if desired, be presented in a pack or dispenser device and/or a kit, which may contain one or more unit dosage forms containing the active ingredients. The pack or kit may, for example, comprise appropriate dispensing devices, such as needles and syringes or blister packs. The pack or dispenser device may be accompanied by instructions for administration.

While this invention has been shown and described with references to particular embodiments, it will be understood by those skilled in the art that various changes in form and detail may be made therein without departing from the spirit and scope of the invention encompassed by the appended claims.

1. A therapeutic composition to treat a proliferative disorder, the composition comprising:
a mCRP antagonist;
a therapeutic ligand capable of activating complement; and
β-glucan.

2. The composition of claim 1 wherein the b-glucan is derived from yeast.

3. The composition of claim 1 wherein the therapeutic ligand is an antibody.

4. The composition of claim 1 wherein the mCRP anatagonist is an antibody.
